(19) United States
(12) Patent Application Publication
(10) Pub. No.: US 2011/0046443 A1
(43) Pub. Date: Feb. 24, 2011

(54) METHOD FOR GUIDING A CAPSULE ENDOSCOPE AND ENDOSCOPE SYSTEM

(76) Inventors: Hironao Kawano, Tokyo (JP); Johannes Reinschke, Nurnberg (DE); Akio Uchiyama, Osone Kohoku-ku Yokohama-shi (JP)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/740,799

(22) PCT Filed: Oct. 14, 2008
(86) PCT No.: PCT/EP08/63778

§ 371 Date: Nov. 5, 2010

(30) Foreign Application Priority Data
Oct. 30, 2007 (DE) 10 2007 051 861.9

(51) Int. Cl. A61B 1/00

(52) U.S. Cl. 600/118

(57) ABSTRACT

In a method and system for guiding a capsule endoscope, the capsule endoscope contains a rotation sensor that measures a rotary position of the endoscope capsule about at least one fixed capsule axis. A control unit calculates a rotary position of the endoscope capsule from a mechanical movement model, measures the rotary position of the endoscope capsule by means of the rotation sensor, compares the measured rotary position with a rotary position calculated for an essentially identical point in time, and adapts the mechanical movement model on the basis of the comparison.

The invention relates to a magnetically guided capsule endoscope or a magnetically guided endoscope capsule as well as to a method for operating the capsule endoscope or, as the case may be, the endoscope capsule system.

A magnetically controlled endoscope capsule is described in DE 101 42 253 C1, for example. The magnetic guidance is achieved by means of magnetic forces based on magnetic gradient fields which act on a permanent magnet in the capsule, the magnetic gradient field being generated by means of an external guidance magnet. The external guidance magnet is preferably an electromagnet, as described in DE 103 40 925 B3 or WO 2006/092421 A1, for example. Alternatively the guidance magnet contains one or more mechanically movable permanent magnets. Alternatively to magnetic guidance by means of magnetic forces, the capsule can be provided externally with a type of thread, as described in US 2003/0181788 A1, and moved through a section of intestine according to the principle of an Archimedes screw, magnetic torques which are produced as a result of the interaction of a rotating external magnetic field with a permanent magnet fixedly incorporated into the capsule acting on the capsule. The magnetization direction of the capsule's permanent magnet lies preferably perpendicular to the longitudinal axis of the capsule. Furthermore, the position and orientation of the capsule can be measured partly electromagnetically, as described in WO 2005/120345 A2 for example, though in that case only 5 of the 6 possible coordinates are measured and a measurement of the angle of rotation around the longitudinal axis of the capsule is not possible.

For precise magnetic guidance, in particular when the guidance magnet generates magnetic fields and/or magnetic gradient fields which are not spatially constant to a large extent in the interior of a working volume, the center-of-mass coordinates and the magnetization direction of the permanent magnet in the capsule, i.e. in particular the angle of rotation about the longitudinal axis of the capsule, must be known precisely at all times. If a sufficiently strong external magnetic field with known orientation is generated at the location of the capsule, the capsule, together with the permanent magnet inside it, rotates about the longitudinal axis until the external magnetic field and the magnetization direction of the permanent magnet coincide once again. If, however, the external magnetic field is not strong enough, a misalignment between the external magnetic field and the magnetization direction of the permanent magnet will persist due to frictional forces which inhibit a free movement of the capsule.

It is the object of the present invention to provide a possibility for precise, in particular magnetic, guidance of a capsule endoscope or an endoscope capsule.

This object is achieved by means of a method according to claim 1 and a device according to claim 12, 13 or 14.

The method for, in particular magnetic, guidance of a capsule endoscope, the endoscope capsule of which comprises a rotation sensor for measuring a rotary position of the endoscope capsule about its longitudinal axis L, comprises the following steps of:
calculating a rotary position of the endoscope capsule from a mechanical movement model;
measuring a rotary position of the endoscope capsule by means of the rotation sensor;
comparing the measured rotary position with a rotary position calculated for an essentially identical point in time;
adapting the mechanical movement model on the basis of the comparison.

By means of said method the rotary position of an endoscope capsule can be determined with a high degree of precision quasi-continuously from the mechanical model or its execution even when an endoscope capsule is used in which the measurement of the rotary position is possible only at comparatively long intervals, typically two or four times per second. In comparison therewith a capsule rotation can be faster, for example up to 8 or 10 revolutions per second. Thus, instead of speeding up the capsule-internal measured value acquisition in a complex and time-consuming manner, possibly while making demands on the power of the capsule-internal electronics as well on the capsule-internal energy consumption, the measured data acquired at only relatively long time intervals are used as input or correction data for the numerical movement model which is comparatively easy to implement and can deliver capsule position data in “real time”.

The method also comprises a calculation, measurement and comparison of further coordinates in addition to the rotary position for the purpose of correcting or adjusting a general capsule position. Thus, the method can also include a calculation of a multi-dimensional, in particular 6D, capsule position from the mechanical movement model; a measurement of a multi-dimensional capsule position (by means of a plurality of capsule-internal and/or capsule-external sensors); a comparison of the measured multi-dimensional capsule position and a capsule position calculated for an essentially identical point in time; and also a calculation of a corrected, in particular 6D, capsule position in the mechanical movement model on the basis of the comparison.

The capsule is moved preferably by means of magnetic guidance. For the purpose of guiding the endoscope capsule the associated endoscope system therefore has a guidance magnet or a magnet system for generating defined magnetic fields at the location of the endoscope capsule. The control for the guidance magnet can be implemented in hardware, firmware, software, or a combination thereof.

If the associated endoscope system uses an electromagnet as a guidance magnet for magnetically guiding the endoscope capsule, a method is preferred in which following the step of calculating the corrected rotary position correspondingly corrected coil currents are set for the guidance electromagnet.

If the associated endoscope system uses a mechanically movable permanent magnet (or a permanent magnet system) as the guidance magnet for magnetically guiding the endoscope capsule, a method is preferred in which following the step of calculating the corrected rotary position a correspondingly corrected position of the permanent magnet or magnets is set.

From the comparison of calculated and measured variables (including variables derived from the measured data) the value of at least one model parameter is preferably also corrected or adjusted, in particular the coefficient of friction of the endoscope capsule. This enables deviations of the mechanical model from reality to be reduced further. Alternatively or in addition, however, other parameters in the mechanical capsule movement model can also be adjusted.

In order to initialize the method, the angle of rotation of the endoscope capsule is preferably aligned into an essentially known position. In the case of a magnetically guided endoscope capsule this is effected by means of a magnet element fixedly installed in the capsule, in particular a permanent magnet, with a fixed magnetization direction, usually in fixed capsule coordinates, preferably in such a way that the angle of rotation of the endoscope capsule is aligned by applying a sufficiently strong capsule-external magnetic field at the location of the capsule, as a result of which a known orientation or alignment of the magnet element on the (capsule-external) magnetic field is achieved at this point in time. This method step is referred to below as the “initial permanent magnet alignment”. Following this, the capsule is moved by means of far lower magnetic fields (and possibly also additional field gradients). Although in this situation the magnetization direction of the magnet element of the capsule is no longer aligned exactly parallel to the (capsule-external) magnetic field vector, the known time characteristic of the magnetic field vector at least indicates the direction and speed of the capsule movement, in particular the “missing coordinate”, i.e. the rotation about an axis of the capsule, preferably the longitudinal axis.

The measurements can drift, however. For this reason it is advantageous if the initial permanent magnet alignment is repeated after a certain number of recordings or measurements.

It is advantageous for the use of conventional endoscope capsules or endoscope capsules that are modified only comparatively slightly if the camera already included in any case is used as the rotation sensor, in particular if its optical axis essentially coincides with the longitudinal axis of the endoscope capsule.

The rotary position is then preferably determined by means of an image comparison of at least two images recorded by the camera at different times, in particular by means of a superposition of the images.

However, the rotation sensor can also have a magnetic field sensor whose field detection direction stands in particular essentially perpendicular to the longitudinal axis L of the capsule endoscope.

It is especially preferred if, at the time an image/a measurement (in the narrower sense) of the rotation sensor is recorded, the time of the recording/measurement is also logged. By superimposing or comparing successive images/measurements it is possible to determine the angle of rotation between e.g. two succeeding images/measurements, and moreover with a knowledge of the rotation of the magnetic field at the times of the two succeeding images/measurements. The direction of rotation can then be incorporated into the information relating to the image/measured value rotation, and the ambiguity in relation to a full revolution (corresponding to modulo 360°) can be removed.

The endoscope system is equipped with a guidance means, in particular a guidance magnet, for guiding an, in particular magnetically guided, capsule endoscope which is configured in order to allow the method according to one of the above claims to be performed.

Particularly advantageously the endoscope capsule is connected to a measured value acquisition device of the endoscope system, the measured value acquisition device being configured to record successive measurement signals from the endoscope capsule, such as the images of a camera or magnetic field measured value signals of the magnetic field sensor, while at the same time recording the time of acquisition. It is also configured for superimposing or comparing the successive outputs of the rotation sensor (images/measured value signals).

The external guidance magnet is preferably coupled to a control unit of the endoscope systems. If the guidance magnet is an electromagnet, the control unit ensures for example that the right currents flow in the individual coils of the electromagnet in the time characteristic. In the case of an external guidance magnet consisting of at least one mechanically movable permanent magnet the control unit ensures the correct movement or positioning of the at least one permanent magnet of the guidance magnet in the time characteristic. Advantageously the control unit is also configured for comparing the experimentally determined rotary position—as described above for example—with a numerically determined rotary position. The numerically determined rotary position can be obtained for example from a numerical model or a numerical simulation which simulates the endoscope capsule in operation.

Preferably the control unit is configured so that the numerical model executes on it.

In general the endoscope capsule can be embodied not only as an autonomous, wireless system, but also as a movable head or movable tip of a catheter or tube. The field of application is not limited to the performance of minimally invasive diagnoses, i.e. visual inspections and/or the taking of samples inside human beings or animals. The endoscope capsule can serve as a therapeutic tool, e.g. for targeted, local application of drugs, or as a diagnostic tool in piping systems.

In addition the endoscope capsule can have for example a lighting device, e.g. an LED, for illuminating the environment, a battery which can be recharged by means of an external alternating field, a sampling device, or additional sensors and/or processing instruments and so forth.

The invention is described schematically in greater detail in the following exemplary embodiment.

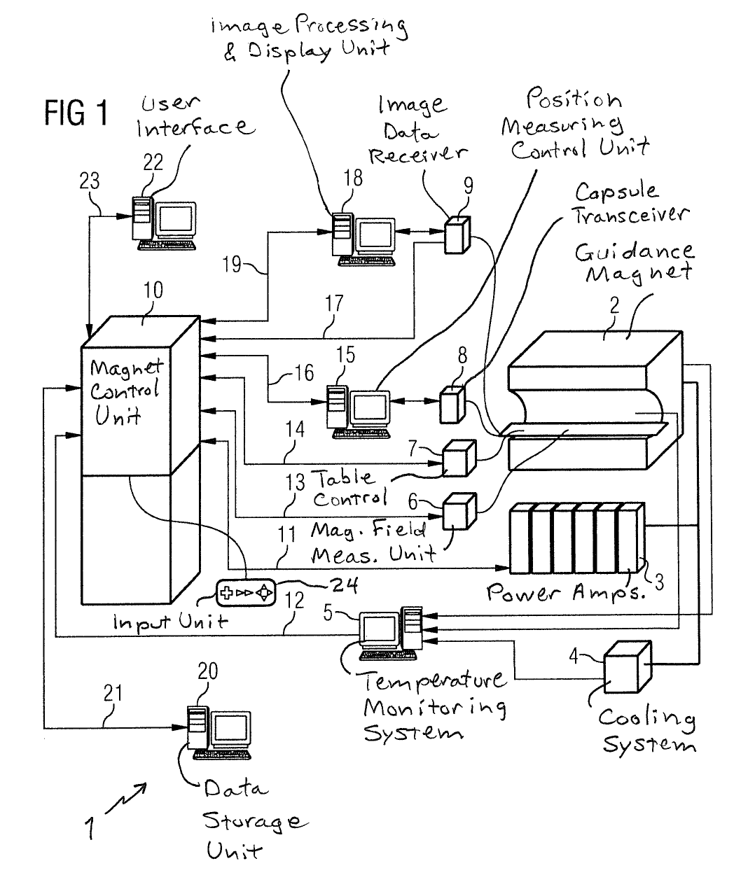

FIG. 1 shows a system for controlling an endoscope;

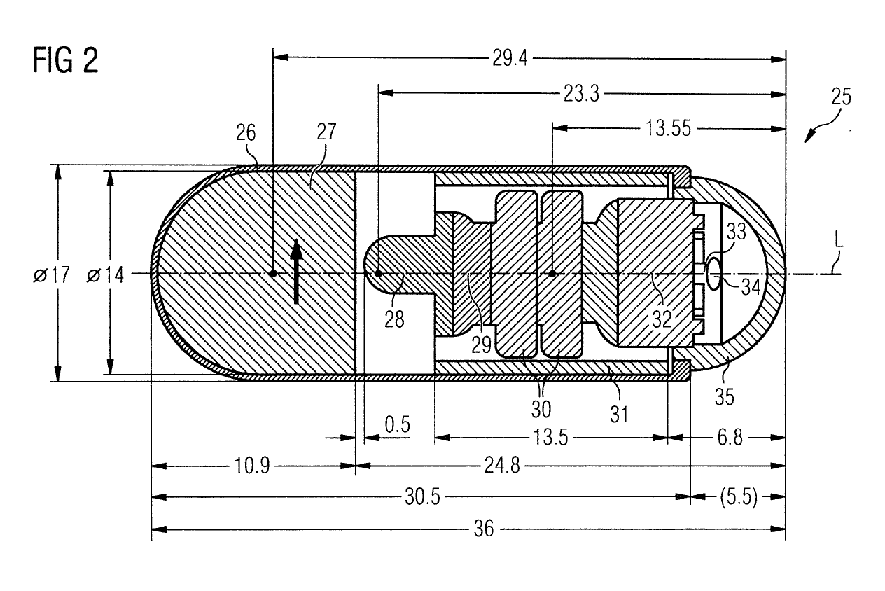

FIG. 2 shows a sectional representation of an endoscope capsule in a side view; and

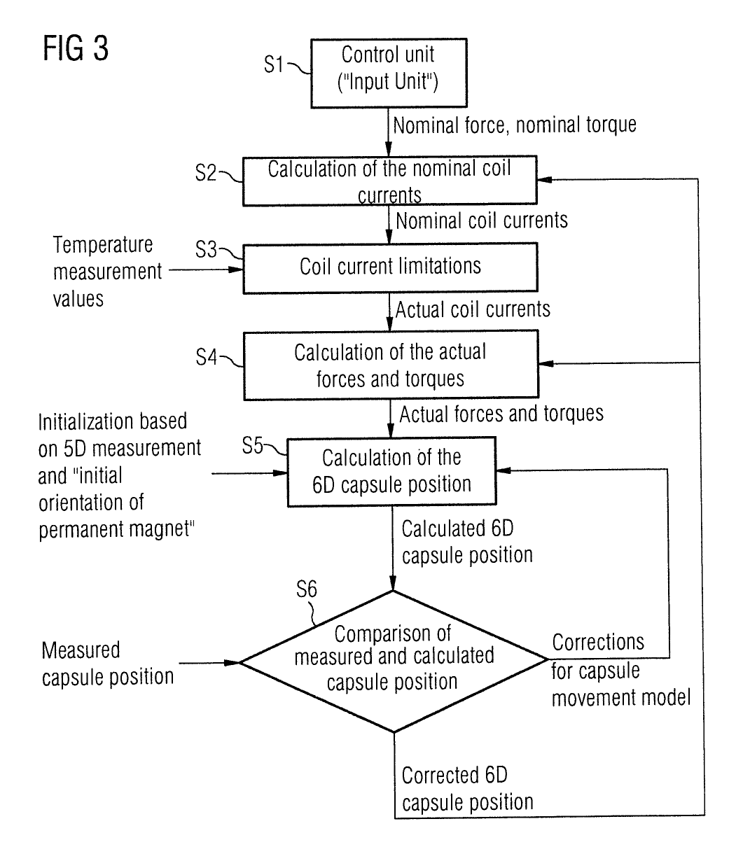

FIG. 3 is a flowchart for controlling the guidance magnet 2 according to FIG. 1.

FIG. 1 shows a layout of a system 1 for controlling an endoscope having a guidance magnet 2 for magnetically guiding an endoscope capsule. For operational purposes the guidance magnet 2 is connected to power amplifiers 3 and a cooling system 4. For temperature monitoring purposes the cooling system 4 and the guidance magnet 2 are additionally connected to a temperature monitoring system 5. Also connected to the guidance magnet 2 are a transmitter/receiver 8 of a position measuring system and an image data receiver 9 as well as optionally a patient table control unit 7 and a magnetic field measuring unit 6.

A guidance magnet control unit 10 serves as a central control unit. The guidance magnet control unit 10 is connected via digital and/or analog data interfaces to the power amplifiers 3, to the temperature monitoring system 5, optionally to a magnetic field measuring unit 6, optionally to a patient table control unit 7, to the position measuring control unit 15 and to the image data receiver 9 and the image processing and display unit 18. The guidance magnet control unit 10 is also coupled via at least one digital data interface to a central data storage unit 20 and to a graphical user interface 22. The digital interfaces can be embodied as an Ethernet connection, CAN bus, RS-232, RS-422, RS-485 or similar. An input unit 24 is part of the guidance magnet control unit 10 or, as the case may be, is connected to the latter.

The control unit 10 is used for controlling the endoscope system 1, in particular for conducting a current through the guidance magnet 10, which as a magnet system can also consist of a plurality of, in particular independently controllable, individual magnets. Toward that end a mechanical movement model of the endoscope or endoscope capsule runs on the control unit 10. At the same time measurement signals for detecting the position of the endoscope capsule are received by means of the transmitter/receiver (transceiver) 8 of the position measuring system and converted by the position measuring control unit 15 into a 5D capsule position. Said 5D capsule position measured values are generated at a clock rate of e.g. 91 Hz and forwarded to the control unit 10. At the same time measured values from the capsule, such as video images and where applicable values of other capsule-internal sensors, are received by the image data receiver 9 at a clock rate of 2 Hz or 4 Hz. The data transfer is effected wirelessly at a carrier frequency e.g. 433 MHz. From the images recorded or received sequentially in time by the endoscope capsule, either the image processing and display unit 18 or the guidance magnet control unit 10 calculates an angle of rotation of the endoscope capsule, in particular using information relating to the movement of the magnetic field at the capsule location between the recording times. The control unit 10 is also configured to derive a correction of the numerically calculated values from a comparison of at least roughly simultaneous measured and calculated values of the rotary position—and possibly of other coordinates—of the endoscope capsule and to convert said correction into a corresponding adjustment of the current or currents through the guidance magnet 2, e.g. by an adjustment of the control signals to the power amplifiers 3. In addition the control unit 10 is configured for adjusting values of model parameters from the comparison of calculated and measured capsule positions in order to provide an even more realistic simulation of the capsule movement, in particular of a coefficient of friction.

FIG. 2 shows an endoscope capsule 25 of a capsule endoscope. A permanent magnet 27 whose magnetization direction is indicated by means of the arrow is accommodated in a housing 26. By means of the permanent magnet 27 the capsule 25 can be aligned with a sufficiently strong external magnetic field for example. Also contained in the housing are a radio-frequency antenna 28 for transmitting and receiving 433 MHz signals and a 433 MHz radio-frequency transmitter 29. Housed adjacent to these are two batteries 30 for supplying power to the capsule 25. A hollow cylindrical LC marker coil 31 for the electromagnetic 5D capsule position measurement is present at a circumferential housing section spaced apart from the permanent magnet 27. At one end the housing also comprises a camera controller 32 which possesses an image compression capability and, coupled thereto, a CMOS sensor 33 having a lens 34 and LEDs for illuminating the field of view. For that purpose the housing 26 is embodied as transparent in the field of view of the CMOS image sensor 33, in this case by means of a see-through cover or dome 35. The optical axis of the camera 33,34,35 or, as the case may be, of the CMOS sensor 33 essentially corresponds to the longitudinal axis L of the endoscope capsule 26. The camera 33,34,35 is in this case additionally used as a rotation sensor.

In other embodiment variants the magnetization direction of the permanent magnet 6 can also be aligned other than perpendicularly to the longitudinal axis L.

The capsule is preferably swallowable or rectally insertable. If the capsule is to be swallowable, smaller external dimensions are preferred than in the case of a capsule that is to be introduced rectally, and moreover a swallowable capsule preferably having a maximum outer diameter of approx. 11 mm and a maximum length of approx. 30 mm.

FIG. 3 shows a flowchart for controlling the guidance magnet 2 from FIG. 1. Except for step S1, this can be implemented in particular in the guidance magnet control unit 10 from FIG. 1, e.g. in software, firmware and/or hardware, or be resident on a data medium, e.g. a hard disk or a DVD.

In a first step S1, a nominal force and a nominal torque are input via the input unit 24 from FIG. 1. From this, in a following step S2, the nominal coil currents that are to flow through the guidance magnet 2 for that purpose are calculated. In a following step S3, the values of the nominal coil currents are used together with acquired temperature measured values for the purpose of determining a limiting of the coil current e.g. in order to avoid overheating. In a step S4, the resulting actual coil currents which are output to the power amplifiers 3 are used for calculating the actual forces and actual torques. The actual forces and actual torques are calculated in the control unit 10 by means of a numerical movement model of the capsule.

In step S5, the actual forces and actual torques are used to calculate the 6D capsule position, i.e. including the rotary position of the capsule about its longitudinal axis. The calculation of the 6D capsule position begins with an initialization from the 5D measurement and the “initial permanent magnet alignment”; for that purpose a magnetic field is generated at the location of the endoscope capsule which is strong enough to align the capsule at least sufficiently accurately with the magnetic field. As a result of the model-based calculation running in the control unit 10 to determine the capsule position in all 6 dimensions a rotary position can also be output in quick succession (“quasi-continuously”). This is of particular advantage because typically a measured value is transmitted from the capsule by means of a rotation sensor for the purpose of determining the rotary position only two to four times per second, whereas the capsule rotates up to eight or ten times per second. The measured value sequence therefore lags behind the sequencing speed required for precise control of the capsule. On the other hand, the values measured externally, e.g. with the aid of the LC marker coil 31 in FIG. 2, can be interrogated more quickly, at a clock rate of e.g. 91 Hz.

In step S6, the 6D capsule position determined with computer support by a model-based calculation in step S5 is compared with the measured capsule position, in particular with approximately simultaneously measured 5D position values of the LC marker coil 31 and rotary position measured values that were determined by means of a rotation sensor in the capsule. The rotary position measured values are produced for example from a comparison of images recorded with a time offset by the capsule camera. From the comparison, on the one hand a corrected 6D capsule position is determined which is used as a correction variable for the calculation of the nominal coil currents in step S2. On the other hand, corrections are determined from the deviations for input into the capsule movement model on which the calculation of the 6D capsule position in step S5 is based.

Between comparisons with the measured and calculated rotary position measured values, the calculated capsule position can moreover be adjusted or corrected only by means of the measured 5D capsule position (without the rotary position), which effects a further increase in guidance accuracy.

The processing of the flowchart according to FIG. 3, i.e. the calculation of 6D capsule positions and the output of actual coil currents to the power amplifiers 3, takes place at a clock rate of e.g. 100 Hz. This clock rate is typically significantly higher than the clock rate of the rotation sensor and also differs from the clock rate of the position measuring control unit.

It goes without saying that the invention is not limited to the exemplary embodiments described.

1. A method for guiding a capsule endoscope, the endoscope capsule (25) of which comprises a rotation sensor (33,34,35) for measuring a rotary position of the endoscope capsule (25) about at least one fixed capsule axis, the method comprising the following steps of:
calculating (S5) a rotary position of the endoscope capsule (25) from a mechanical movement model (S5);
measuring a rotary position of the endoscope capsule (25) by means of the rotation sensor (33,34,35);
comparing (S6) the measured rotary position with a rotary position (S6) calculated for an essentially identical point in time;
adapting (S7) the mechanical movement model on the basis of the comparison.

2. The method as claimed in claim 1 for magnetically guiding a capsule endoscope, the method additionally having the following step after the step of calculating the rotary position:
setting coil currents in a guidance magnet (2) in the form of an electromagnet for guiding the capsule endoscope on the basis of the calculated rotary position of the capsule endoscope.

3. The method as claimed in claim 1 for magnetically guiding a capsule endoscope, the method additionally having the following step after the step of calculating the rotary position of the capsule endoscope:
setting a corrected position of a guidance magnet (2) in the form of a mechanically movable permanent magnet for guiding the capsule endoscope on the basis of the calculated rotary position of the capsule endoscope.

4. The method as claimed in claim 1 or 2, the method additionally having the following step after the comparison step (S6):
correcting (S7) at least one model parameter, in particular a coefficient of friction between the endoscope capsule (25) and its environment, in the mechanical movement model on the basis of the comparison.

5. The method as claimed in one of the preceding claims, the method having the following step before the calculation step (S5):
aligning the angle of rotation of the endoscope capsule (25).

6. The method as claimed in claim 5, wherein the endoscope capsule (25) has a fixed capsule-internal permanent magnet (27) with predetermined magnetization direction and the step of aligning the angle of rotation of the endoscope capsule (25) presents itself as follows:
aligning the angle of rotation of the endoscope capsule (25) by application of an appropriately strong magnetic field.

7. The method as claimed in claim 6, wherein the step of aligning the rotary position of the endoscope capsule (25) is repeated by application of an appropriately strong magnetic field at predetermined time intervals.

8. The method as claimed in one of the preceding claims, wherein the rotation sensor has a camera (33,34,35) and the step of measuring the rotary position of the endoscope capsule (25) includes an image comparison of at least two images recorded by the camera (33,34,35) at different times, in particular by means of a superposition of the images.

9. The method as claimed in claim 8, wherein in order to eliminate a modulo(2π) ambiguity in the determination of the rotary position between two images a rotary direction is determined from the rotation of the magnetic field between the recording times of the images.

10. The method as claimed in claim 8 or 9, wherein an optical axis of the camera (33,34,35) lies essentially along the longitudinal axis (L) of the capsule endoscope (1).

11. The method as claimed in one of claims 1 to 6, wherein the rotation sensor has a magnetic field sensor whose field detection direction stands essentially perpendicular to the longitudinal axis (L) of the capsule endoscope (1).

12. An endoscope system (1) having a guidance magnet (2) for guiding a capsule endoscope (25) which is configured for allowing the method as claimed in one of the above claims to be performed.

13. An endoscope system (1) having a guidance magnet (2) in the form of an electromagnet for guiding a capsule endoscope (25) which is configured for allowing the method as claimed in claim 2 to be performed.

14. An endoscope system (1) having a guidance magnet (2) in the form of a mechanically movable permanent magnet for guiding a capsule endoscope (25) which is configured for allowing the method as claimed in claim 3 to be performed.
